(19) United States
(12) Patent Application Publication
(10) Pub. No.: US 2017/0000937 A1
(43) Pub. Date: Jan. 5, 2017

(54) Dialysis Apparatus Comprising a Dialyzer

(71) Applicant: NEPHRO-SOLUTIONS AG, Hamburg (DE)

(72) Inventor: Oliver Gottschalk, Hamburg (DE)

(21) Appl. No.: 15/113,162

(22) PCT Filed: Jan. 6, 2015
(86) PCT No.: PCT/EP2015/050083

§ 371 Date: Jul. 21, 2016

(30) Foreign Application Priority Data
Jan. 21, 2014 (DE) 10 2014 000 737.5

(51) Int. Cl. A61M 1/16; B01D 63/04; B01D 69/08; A61M 1/36; B01D 69/02

(52) U.S. Cl. CPC A61M 1/1625; A61M 1/3672; B01D 69/02; B01D 69/08; B01D 63/04; A61M 2205/7509; A61M 2205/7518; B01D 2325/022

(57) ABSTRACT

The invention relates to a dialysis apparatus comprising a dialyzer (4) that includes a cluster of capillary membranes (8), each of which has an outer face (13) and an inner face (14), the mean pore size being larger in the area of the outer face (13) than in the area of the inner face (14), further comprising a blood circulation system (6) and a dialysate circulation system (7), the blood circulation system (6) extending along the outer faces (13) and the dialysate circulation system (7) extending along the inner faces (14).

The invention concerns a dialysis apparatus comprising a dialyser with a cluster of capillary membranes, each of which has an outer face and an inner face and a mean pore size which is larger in the region of the outer face than in the region of the inner face, a blood circulation system and a dialysate circulation system.

Dialysis machines have been known for a long time in the prior art. Worldwide, there are presently around 2.5 million dialysis patients who owe their life to kidney replacement therapy. To perform kidney replacement therapy, dialysers are largely used with capillary membranes which, in the basic treatment with so-called low flux dialysers, remove from the blood the low molecular, toxic, urinary-excreted substances, such as for example urea and creatinine.

As an alternative, high flux membranes are used which, because of their larger pore sizes in the membrane structure, also allow part of the retention toxins with mean molecular weights to pass through, e.g. β2 microglobulin (β2-M). Whereas in low flux dialysis, blood cleansing mainly takes place on the basis of diffusive processes, high flux dialysis profits in particular from convective (solvent drag) processes as well as having large pore sizes.

The wall of modern, fully synthetic capillary membranes is constructed asymmetrically. Capillary membranes made of polysulfone have on the inside a thin membrane structure a few micrometres thick, which is mechanically fixed by a 40 μm thick, stabilising supporting structure which is increasing porous towards the outside.

Because of its polymer composition, this supportive layer is able to absorb different molecules up to endotoxins, bacteria and viruses.

Disadvantageously and because of their corresponding size, conventional dialysis machines do not filter viruses and bacteria out of the blood.

It is an object of the invention to provide a dialysis apparatus which avoids the above-mentioned disadvantage.

The invention uses the idea of reversing the conventional ultrafiltration direction along the capillary membranes of the dialyser. For this, according to the invention, the blood circulation system is guided along the outer face of the capillary membrane and the dialysis circulation system along the inner faces of the capillary membrane. Advantageously, the blood therefore flows along the outer face which has the larger pores. In the course of ultrafiltration from outside to inside, viruses, bacteria and endotoxins can be captured in the larger pores and extracted from the patient's blood by adsorption or size exclusion. The actual dialysis process, which takes place via the inner face with smaller pore size, is carried out in principle conventionally.

Favourably, a mean pore size on the outer face of the capillary membrane is between 1 and 4 μm, and favourably a mean pore size of an inner face of a capillary membrane is less than 5 nm. Bacteria and viruses have a size between 40 nm and a few microns, and thus via the large pores of the outer face enter the interior of the capillary membrane wall where they are captured. Therefore they do not remain in the blood circulation system, as in conventional dialysis, but are absorbed from this.

Favourably, in cross-section perpendicular to the longitudinal direction, the capillary membranes have a circular inner face and a circular outer face; preferably, the capillary membranes are formed tubular along their entire longitudinal extension, and are formed circular in cross-section in both the inner and outer faces along their entire longitudinal extension. The capillary membranes are easy to produce.

The capillary membranes are part of a dialyser which is formed by a housing on the outside. The cluster of capillary membranes is arranged in the housing which is formed tubular in the longitudinal direction. The capillary membranes are preferably arranged parallel to each other and cast in a holder at their open ends, whereby a blood compartment is also formed between the outer face of the capillary membranes and the inner wall of the housing, i.e. forming part of the blood circulation system, whereas the lumens of the capillaries form a portion of the dialysate circuit and constitute a dialysate compartment. The dialysate compartment and blood compartment are separated from each other by the semipermeable capillary membranes. The actual ultrafiltration process takes place through the capillary membranes.

Preferably, in the housing extending in the longitudinal direction, the cluster of capillary membranes is arranged running in the longitudinal direction, and the blood compartment has two second female connector types and the dialysate compartment has two second male connector types arranged on respective opposite ends in the longitudinal direction of the respective compartment. The two female connector types each cooperate with a blood circuit connector arranged on a respective end of a blood circuit hose.

Preferably, two second male connectors are arranged on the dialyser housing and a second adapter type is placed on each of the two second male connectors, said adapter types after application each having a free second female connector intended for connection to a blood circuit adapter; also two second female connectors are arranged on the housing of the dialyser and a first adapter type is placed on each of the two second female connectors, said adapter types each having a free second male connector type intended for connection to a dialysate circuit.

Due to the first and second adapter types, the blood circuit adapters and the dialysate circuit adapters of conventional dialysis machines can be retained, and at the same time the blood compartment and dialysate compartments can be exchanged to achieve the reversal of the ultrafiltration direction essential to the invention.

The invention is described as an example with reference to an exemplary embodiment in seven figures. These show:

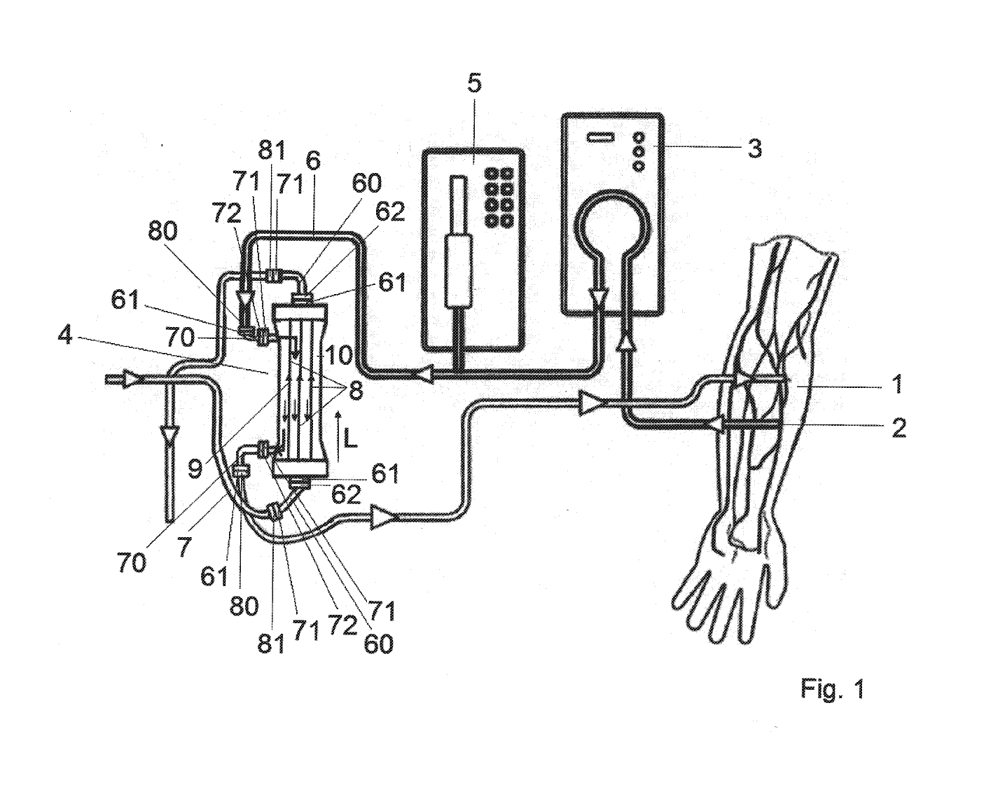

FIG. 1 a dialyser according to the invention connected to a patient,

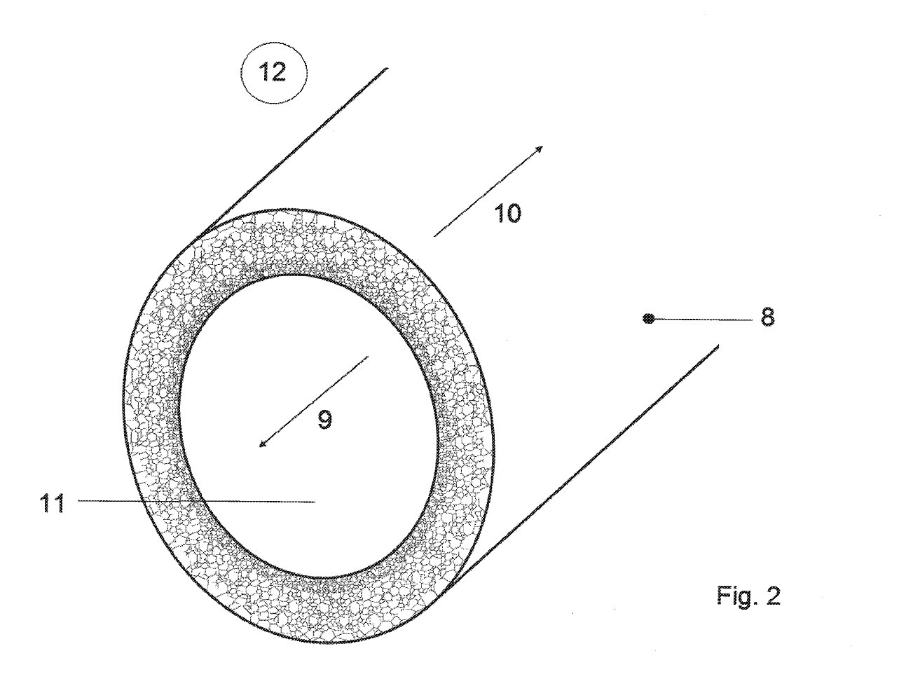

FIG. 2 a perspective view of a capillary membrane,

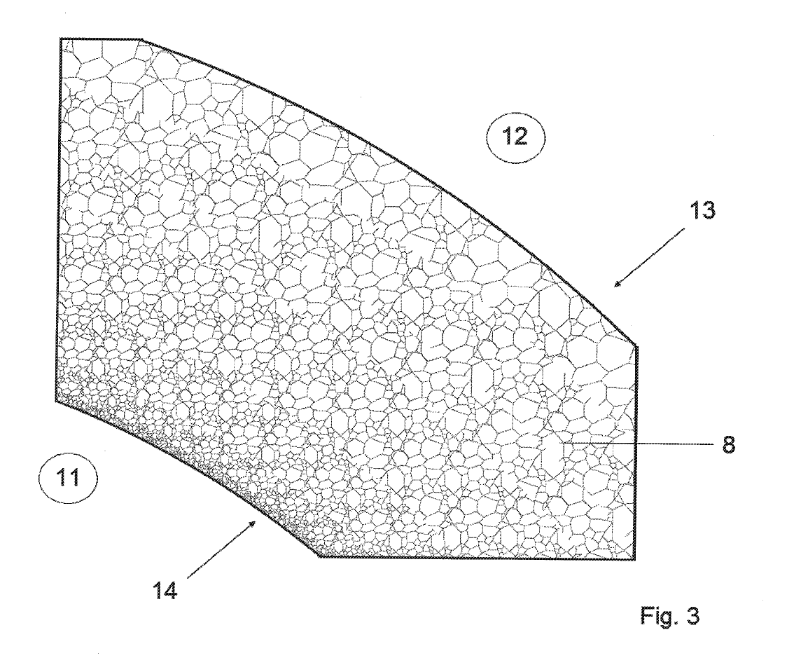

FIG. 3 an extract of the capillary wall with asymmetrically distributed pores,

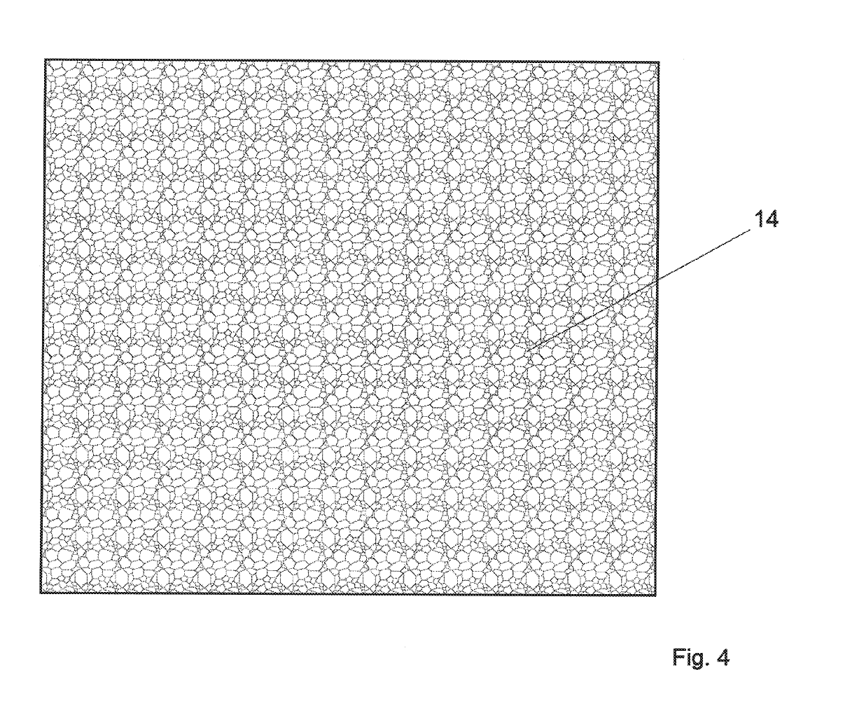

FIG. 4 a view of an inner face of the capillary membrane in FIG. 2,

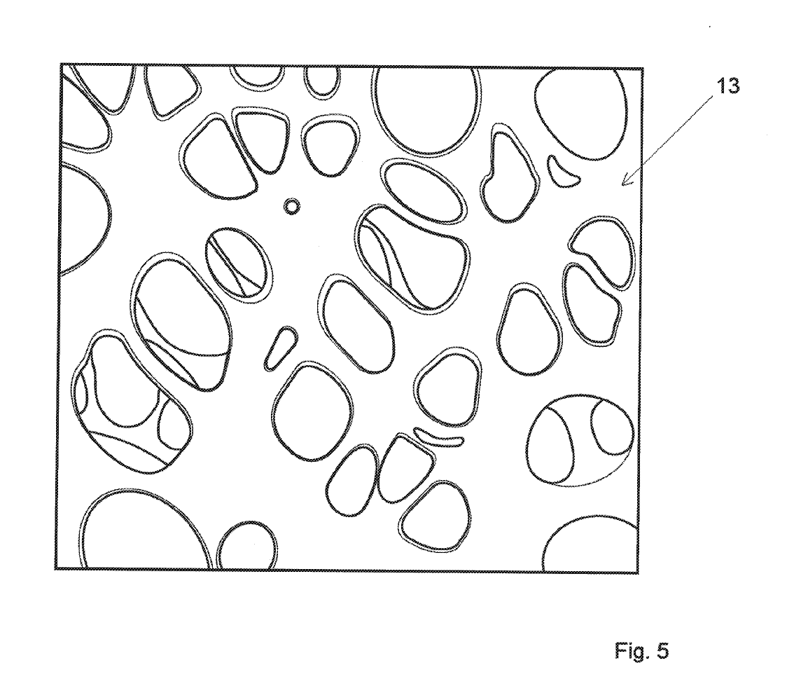

FIG. 5 a view of an outer face of the capillary membrane in the same scale as FIG. 4, and

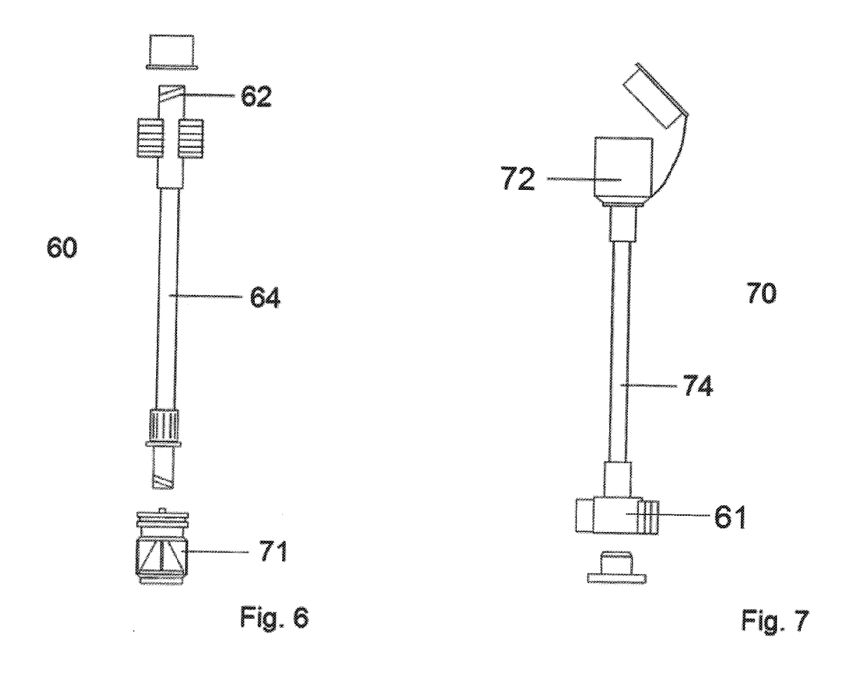

FIG. 6 an adapter of a first type, and

FIG. 7 an adapter of a second type.

FIG. 1 shows diagrammatically the principal structure of a dialysis apparatus for performance of a haemodialysis on a patient. The patient's blood 10 is cleansed extracorporeally. FIG. 1 shows the lower arm 1 of the patient. The blood 10 is extracted from the patient's lower arm 1 by means of a vascular access, conveyed by means of a blood pump 3 via the access 2, and supplied to a dialyser 4. The blood 10 taken from the patient is also mixed with an anti-coagulant in a supply device 5, and the blood 10 enriched with the anti-coagulant is pumped into the dialyser 4 and cleansed in the dialyser 4. The dialyser 4 serves as the actual “artificial kidney”, which washes the waste products out of the through-flowing blood 10 of the patient and also extracts water from the through-flowing blood 10.

The dialyser 4 comprises a portion of an extracorporeal blood circulation system 6 and a portion of a dialysate circulation system 7 which is separate therefrom. Blood 10 is supplied to the dialyser through the blood circuit 6, and dialysis fluid, also called dialysate, is supplied via the dialysate circuit 7. The portion of the blood circuit 6 and the portion of the dialysate circuit 7 are in contraflow in the dialyser 4 and separated from each other by semipermeable capillary membranes 8. The semipermeable capillary membranes 8 are shown in FIG. 2 in a perspective view. In FIG. 1, the capillary membranes 8 are depicted by three continuous lines marked with a flow direction. In the dialyser, around 10,000 capillary membranes are arranged next to each other, largely parallel to each other. Blood 10 from the portion of the blood circuit 6 washes over each of the capillary membranes 8.

The dialyser 4 shown in FIG. 1 substantially comprises a plurality of capillary membranes 8 arranged parallel to each other in a longitudinal direction L. The capillary membranes 8 are small tubes of the diameter of a hair, which have an inner diameter of between 150 μm and 240 μm, and an outer diameter of between 200 μm and over 300 μm. The capillary membranes 8 are arranged parallel to each other, preferably without direct contact with each other, in the dialyser 4. A dialysate 9 from the dialysate circuit 7 flows through each lumen 11 of each of the capillary membranes 8, i.e. through the respective free inner tubes of the capillary membranes 8, in the longitudinal direction L of each of the capillary membranes 8. In an outer chamber 12 of each of the capillary membranes 8, which surrounds the capillary membranes 8 as shown in FIGS. 2 and 3, the blood 10 of the blood circuit 6 flows over the outside of each of the capillary membranes 8 in the opposite direction, i.e. opposite the longitudinal direction L. The capillary membranes 8 are each configured semipermeable.

FIG. 3 shows an extract of the wall of the capillary membranes 8 in FIG. 2. The wall of the capillary membrane 8 preferably consists of fully synthetic polymers and is structured asymmetrically in the radial direction. In the longitudinal direction L however, the capillary membranes 8 are formed substantially translation-invariant. The capillary membranes 8 have an outer face 13 and an inner face 14, wherein the inner face 14 has a very fine membrane structure, i.e. a small membrane thickness of around 1 μm and a mean pore size of <5 Nm, whereas the capillary membrane wall 8 has a pore size which increases in the direction of the outer surface 13 and the pore size here is preferably 1-4 μm. The inner face 14 of the capillary membrane 8 is also mechanically fixed by the ever more porous supporting structure. The different pore sizes of the outer face 13 and in the face 14 are evident from a comparison of FIGS. 3 and 4, depicted on the same scale.

In contrast to conventional dialysis machines, according to the invention the blood circuit 6 and the dialysate circuit 7 are exchanged. In a conventional dialysis treatment, in the known fashion the blood 10 is conducted through the lumen 11 of the capillary membranes 8 and the dialysate 9 is conducted in contraflow on the outside around the capillary membranes 8. According to the invention, the procedure is precisely reversed, in that dialysate 9 flows through the lumen 11 of the capillary membranes 8 and the blood 10 flows around the capillary membranes 8 on the outside. Thus the direction of the ultrafiltration is now reversed and takes place from the outside in the direction of the lumen 11 of the capillary membranes 8. As a consequence of the reversed direction of ultrafiltration, in comparison with conventional dialysis, a larger absorption area of around 1500 m² is available. Here the very large inner face of the capillary membranes 8 formed by the pores is determined by estimation. The large absorption face in the wall of the capillary membranes 8 may be used to remove large-molecular toxins, bacteria or viruses which remain in the pores of the capillary membrane 8 during ultrafiltration since the porosity becomes finer from outside to inside. At the same time, dialysis takes place as before in the capillary membranes 8 by diffusion and convection depending on the dialyser type and method.

Advantageously, a conventional dialyser 4 may be used as part of the dialysis apparatus according to the invention. For this, the dialyser 4 is expanded by two first and two second adapter types 60, 70 in FIGS. 6 and 7. The dialyser 4 has two second female connector types 61 which are provided on the ends of the dialyser 4 in FIG. 1 and are in fluid-conductive connection with the lumen 11 of the capillary membranes 8. On the cylindrical outer wall, the dialyser 4 has two second male connector types 71.

The first adapter type 60 has a first male connector type 62 and a second male connector type 71. The first male connector type 62 and second male connector type 71 are arranged at different ends of a first hose 64 and lie opposite each other.

The second adapter type 70 has a first female connector type 72 and a second female connector type 61. The first female connector type 72 and second female connector type 61 are arranged opposite each other at different ends of a second hose 74.

The second male connector types 71 arranged on the outer wall of the dialyser 4, and the second male connector types 71 on the first adapter type 60, are not necessarily identical in structure but merely identical in function, in the sense that they form a fluid-tight connection with a first female connector type 72. The same also applies to the second female connector type 61 which must form a fluid-tight connection with the first male connector type 62.

A first adapter type 60 with its first male connector type 62 is placed on each of the second female connector types 61 of the dialyser 4. The second female connector type 61 has an internal thread; the first male connector type 62 has an external thread.

A second adapter type 70 with its first female connector type 72 is placed on each second male connector type 71. A plug-type connection is created.

The two second female connector types 61 which depart from the second adapter type 70 are each connected to a blood circuit adapter 80. The two second male connector types 71 are each connected to a dialysate circuit adapter 81. The two adapter types 60, 70 thus exchange the connector types of conventional dialysers 4.

### LIST OF REFERENCE NUMERALS

L Longitudinal direction

1 Lower arm

2 Access

3 Blood pump

4 Dialyser

5 Supply device for anticoagulant

6 Blood circulation system

7 Dialysate circulation system

8 Capillary membranes

9 Dialysate

10 Blood

11 Lumen

12 Outer chamber

13 Outer face

14 Inner face

60 First adapter type

61 Female connector of second type

62 Male connector of first type

64 First hose

70 Second adapter type

71 Male connector of second type

72 Female connector of first type

74 Second hose

80 Blood circuit adapter

81 Dialysate circuit adapter

1. Dialysis apparatus comprising a dialyser (4) with a cluster of capillary membranes (8) each having an outer face (13) and an inner face (14) and a mean pore size which is larger in the region of the outer face (13) than in the region of the inner face (4), a blood circulation system (6) and a dialysate circulation system (7), characterised in that the blood circulation system (6) is guided along the outer faces (13) and the dialysate circulation system (7) is guided along the inner faces (14).

2. Dialysis apparatus according to claim 1, characterised in that a mean pore size on the capillary outer face (13) is between 1-4 μm.

3. Dialysis apparatus according to claim 1, characterised in that a mean pore size on the capillary inner face (14) is less than 5 nm.

4. Dialysis apparatus according to claim 1, characterised in that the capillary membranes (8) in cross-section have a circular inner face (14) and a circular outer face (13).

5. Dialysis apparatus according to claim 1, characterised in that the capillary membranes (8) surround lumens (11) which are part of the dialysate compartment, and a chamber (12) surrounding the capillary membranes (8) on the outside is part of the blood compartment.

6. Dialysis apparatus according to claim 1, characterised by a housing extending in the longitudinal direction (L) in which the cluster of capillary membranes (8) is arranged running in the longitudinal direction (L), and the blood compartment has two second female connector types (61) and the dialysate compartment has two second male connector types (71).

7. Dialysis apparatus according to claim 1, characterised in that two second male connector types (7) are arranged on the housing and connected to the blood compartment, and an adapter of the second type (70) is placed on the outside on each of the two second male connector types (71), and two second female connector types (61) are arranged on the housing which are connected to the dialysate compartment, and an adapter of the first type (60) is placed on each of the two second female connector types (61), each said adapter (60) having two second male connector types (71).
